(19) United States
(12) Patent Application Publication
(10) Pub. No.: US 2009/0065369 A1
(43) Pub. Date: Mar. 12, 2009

(54) CATALYTIC NANOCARBON ELECTRODES FOR BIOSENSORS

(75) Inventors: Keith J. Stevenson, Austin, TX (US); Jennifer L. Lyon, Austin, TX (US)

Correspondence Address: SCHWABE, WILLIAMSON & WYATT, P.C.;PACWEST CENTER, SUITE 1900, 1211 SW FIFTH AVENUE, PORTLAND, OR, 97204, US

(73) Assignee: The Board of Regents, The University of Texas System, Austin, TX (US)

(21) Appl. No.: 12/207,966

(22) Filed: Sep. 10, 2008

Related U.S. Application Data
(60) Provisional application No. 60/993,201, filed on Sep. 10, 2007.

(51) Int. Cl. G01N 27/26; C12Q 1/26; G01N 27/327; C12Q 1/54

(52) U.S. Cl. 205/777.5; 435/25; 435/14; 204/403.01; 204/403.15; 204/403.14

(57) ABSTRACT

Embodiments of the present invention provide a method for detecting a composition comprising (a) providing a first composition which reacts with an oxidase to generate a second composition; (b) providing nitrogen-doped nanocarbons; and (c) detecting the first composition with the nanocarbons. Devices and systems containing such nitrogen-doped nanocarbons are also provided.

### CROSS REFERENCE TO RELATED APPLICATIONS

The present application claims priority to U.S. Provisional Patent Application No. 60/993,201, filed Sep. 10, 2007, entitled “Catalytic Nanocarbon Electrodes for Peroxide-Based Biosensors,” the entire disclosure of which is hereby incorporated by reference in its entirety.

### GOVERNMENT INTERESTS

This invention was made with Government support under Grant/Contract No. CHE-0134884 awarded by the National Science Foundation. The Government has certain rights in the invention.

### TECHNICAL FIELD

Embodiments of the present invention relate generally to nanocarbons, and more particularly to the use of doped nanocarbons in biosensors.

### BACKGROUND

Hydrogen peroxide (H₂O₂) is produced as a byproduct of many oxidase-substrate interactions involving such physiologically important molecules as glucose and cholesterol. Therefore, the detection and quantification of H₂O₂ has become the basis of many biosensing strategies, including electrochemical biosensing.

Biosensors developed for glucose or cholesterol detection typically utilize the respective oxidases of these substrates, which catalytically generate hydrogen peroxide (H₂O₂) upon interaction with them. This enzymatically generated H₂O₂ may then be detected by direct electrochemical H₂O₂ oxidation. H₂O₂ may also be detected through enzymatic H₂O₂ reduction incorporating an electrochemically detectable peroxidase, such as horseradish peroxidase (HRP).

### BRIEF DESCRIPTION OF THE DRAWINGS

Embodiments of the present invention will be readily understood by the following detailed description in conjunction with the accompanying drawings. Embodiments of the invention are illustrated by way of example and not by way of limitation in the figures of the accompanying drawings.

FIG. 1 is a depiction of glucose detection at a glassy carbon (GC) electrode with co-immobilized glucose oxidase (GOx) and nitrogen doped carbon nanotubes (N—CNT) in accordance with various embodiments of the present invention;

FIG. 2 is a group of representative cyclic voltammograms (CVs) for the reduction of oxygen at undoped CNT- and N—CNT-modified GC electrodes immersed in pH 6.00±0.03, 0.1 M Na₂HPO₄ in accordance with various embodiments of the present invention; the vertical line on each CV denotes the potential at which the response curves in FIGS. 3 and 4 were collected;

FIG. 3 is a response curve for 25 μM injections of H₂O₂ at N—CNT-modified GC electrodes immersed in pH 6.00±0.03, 0.1 M Na₂HPO₄ in accordance with various embodiments of the present invention; and

FIG. 4 is a response curve for 50 μM injections of D-glucose at a N—CNT/GOx-modified GC electrode immersed in pH 6.00±0.03, 0.1 M Na₂HPO₄ in accordance with various embodiments of the present invention.

### DETAILED DESCRIPTION OF EMBODIMENTS OF THE INVENTION

In the following detailed description, reference is made to the accompanying drawings which form a part hereof, and in which are shown by way of illustration embodiments in which the invention may be practiced. It is to be understood that other embodiments may be utilized and structural or logical changes may be made without departing from the scope of the present invention. Therefore, the following detailed description is not to be taken in a limiting sense, and the scope of embodiments in accordance with the present invention is defined by the appended claims and their equivalents.

Various operations may be described as multiple discrete operations in turn, in a manner that may be helpful in understanding embodiments of the present invention; however, the order of description should not be construed to imply that these operations are order dependent.

The description may use perspective-based descriptions such as up/down, back/front, and top/bottom. Such descriptions are merely used to facilitate the discussion and are not intended to restrict the application of embodiments of the present invention.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the phrases “in an embodiment,” or “in embodiments,” which may each refer to one or more of the same or different embodiments. Furthermore, the terms “comprising,” “including,” “having,” and the like, as used with respect to embodiments of the present invention, are synonymous.

Embodiments of the present invention provide doped nanocarbons for detection of H₂O₂ as an indicator of the presence of and/or the concentration of one or more substrates/analytes, such as glucose, in a sample.

In an embodiment, a method for detecting a substrate is provided comprising providing a first composition that reacts with an oxidase to generate a second composition; and detecting the first composition with nitrogen-doped nanocarbons.

In an embodiment, a device for sensing a first composition that reacts with an oxidase to generate a second composition is provided. Such a device may, in an embodiment, include a first electrode and a plurality of nitrogen-doped nanocarbons disposed on a surface of the first electrode.

While biosensors exist that electrochemically detect H₂O₂ produced from substrate-oxidase interactions through oxidation at an electrode (such as a platinum electrode), this approach may encounter a number of drawbacks, including poor selectivity, low sensitivity, and high susceptibility to electrode fouling at the electrode. Moreover, the electrochemical detection of H₂O₂ generated at a Pt electrode at physiological pH values (that is, at pH values within the range of about 6.5 to about 7.5) is mechanistically complex, and occurs at potentials (+0.4 to +0.7 vs. Ag/AgCl for oxidation) where other electroactive species, such as uric acid and ascorbic acid, create interference.

In addition, detection schemes that rely on the enzymatic reduction of H₂O₂ through a peroxidase have their own set of drawbacks. In addition to being expensive, peroxidases denature easily upon adsorption to many electrode surfaces, and tend to be active toward H₂O₂ only within very limited pH ranges.

Alternatively, various embodiments of the present invention may provide methods and devices for detecting H₂O₂ that are suitable for use in biosensor applications, offer good selectivity and sensitivity, are not susceptibility to electrode fouling, may be operated at potentials where other electroactive species do not interfere, and/or that may be utilized over a broad pH range.

In an embodiment, H₂O₂ may be electrochemically detected by its decomposition at the surface of certain nanocarbons. In an embodiment, H₂O₂ may be electrochemically detected by its decomposition at the surface of certain nitrogen-doped nanocarbons. In an embodiment, nitrogen-doped nanocarbons may be selectively doped carbon nanotubes (N—CNTs), whether single walled or multi-walled.

While carbon nanotubes are discussed herein, other nanocarbon based structures may be utilized in embodiments, such as graphene, buckyballs, buckytubes, fullerenes, etc.

In embodiments, N—CNTs may be grown or formed using one or more of a variety of known or later developed techniques, such as arc discharge, chemical vapor deposition, and laser ablation.

In an embodiment, N—CNTs may be grown via chemical vapor deposition (CVD) using pyridine and ammonia gas precursors. In an embodiment, N—CNTs may be drop-cast at the surface of glassy carbon (GC) electrodes. In accordance with an embodiment, selective doping of carbon nanotubes with nitrogen provides high surface area materials which catalytically decompose hydrogen peroxide. In an embodiment, these materials may be used in real-time, quantitative electrochemical biosensing schemes that rely on the detection of H₂O₂ as a byproduct of oxidase-substrate interactions.

In an embodiment, N—CNTs may be used to induce the decomposition of H₂O₂ into O₂, which is then electrocatalytically reduced at the N—CNTs. Therefore, the consumption of H₂O₂ generated via oxidase-substrate reactions at N—CNT electrodes may be monitored. Since the applied potential in this scheme (+0.2 V vs. Ag/AgCl) is much lower than that required for H₂O₂ oxidation at Pt, interference created by other electroactive species, such as uric acid or ascorbic acid, may be comparatively reduced or eliminated.

In an embodiment, H₂O₂ produced in oxidase-substrate interactions may be detected directly and electrochemically at the N—CNT-GC electrode via a current response corresponding to the decomposition of H₂O₂ into O₂, which is catalytically reduced by the N—CNTs. Since carbon is an inherently good electrode material, the likelihood of electrode fouling in embodiments is reduced. In addition, in an embodiment, peroxide sensing occurs directly at the surface of the nanocarbons, without requiring the use of linking or modifying chemistries. In an alternative embodiment, linking or modifying chemistry may be used, as desired.

In an embodiment, the utilization of N—CNTs in a sensing scheme eliminates the need for a peroxidase enzyme for H₂O₂ detection. This is advantageous in that N—CNTs are considerably less expensive and much more robust than peroxidases, and are reactive toward H₂O₂ over a much broader pH range than peroxidases. By contrast, peroxidases are limited in sensing applications by their tendency to denature if exposed to pH levels outside of a narrow physiological range. In an alternative embodiment, a peroxidase enzyme may be used in conjunction with an N—CNT, as desired.

Since the sensing schemes disclosed herein operate by detecting the H₂O₂ generated from enzyme-substrate interactions, any biological substrate that produces H₂O₂ as a byproduct in its enzymatic oxidation (via oxidase-substrate interactions) may potentially be detected or quantified using these schemes. Examples of such substrate-oxidase couples include pyruvate-pyruvate oxidase, lactate-lactate oxidase, glutamate-glutamate oxidase, ascorbate-ascorbate oxidase and glucose-glucose oxidase.

Embodiments may also be provided in which electrodes coupled to N—CNTs are incorporated into a substrate/analyte sensing system. For example, a substrate sensing system may have an integrated mechanism or may be further coupled to a mechanism for sampling blood from an individual. In an embodiment, the electrode may be coupled to various electronic components to process the signal/current generated by the sensed substrate. Such electronic components may comprise a processor, memory, transmitter, receiver, transceiver, battery, display, etc. In embodiments, sensing electrodes may be incorporated into implantable, semi-implantable, or ex-vivo devices for detecting/monitoring one or more substrates in a body.

The devices and compositions disclosed herein will now be further described with respect to the following specific, non-limiting examples.

In accordance with an exemplary embodiment, N—CNTs were prepared via a floating catalyst chemical vapor deposition process using a ferrocene growth catalyst and pyridine carbon-nitrogen source as described in Maldonado, S.; Morin, S.; Stevenson, K. J., Carbon, 2006, 44, pp. 1429-1437. Briefly, 1.0 mL of a 20 mg/mL ferrocene-pyridine mixture was injected at 0.1 mL/min into a dual-zone quartz tube furnace. The mixture was volatilized at 150° C. in the first zone and then carried downstream to the second zone by Ar carrier gas at a flow rate of 575 sccm. Upon reaching the second zone, the mixture was pyrolyzed at 800° C., respectively, resulting in the base-catalyzed growth of multi-walled N—CNTs from iron nanoparticle nucleation sites. The N—CNTs were deposited along the walls of the quartz tube and were collected after cooling the tube to room temperature under Ar. The nominal lengths and diameters of the as-prepared N—CNTs were 10 μm and 20-40 nm, respectively. N—CNTs were stored in airtight vials prior to electrochemical analysis.

For electrochemical analysis, N—CNTs were drop-cast onto a 0.5 cm diameter GC electrode (PINE Instruments AFE2MO50GC). Before each experiment, the GC electrode was polished successively with 0.3 and 0.05 μm alumina slurries on microcloth (Buehler) to a mirror finish and sonicated in ultrapure H₂O for 15 minutes. For adherence of N—CNT solutions to the GC surface during rotating disk experiments, a 5 wt % NAFION® persulfonated ion exchange polymer solution (obtained commercially from Sigma-Aldrich, Inc., St. Louis, Mo.) was modified with tetrabutylammonium bromide (referred to herein as TBABr-Nafion) using the methods disclosed in Minteer, S. et al., J. Membrane Sci., 2003, 213, pp. 55-66 and Minteer, S. et al., J. Membrane Sci., 2006, 282, pp. 276-283. The TBABr-Nafion solution was diluted to 0.075 wt % using absolute ethanol, and N—CNTs were suspended in the solution at a concentration of 5 mg/mL.

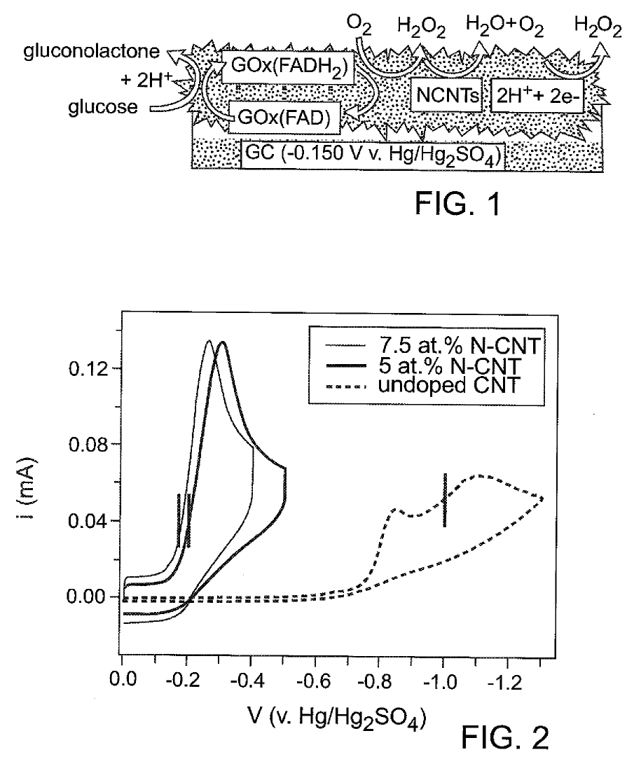

For H₂O₂ calibration standards and glucose determination using soluble glucose oxidase (GOx; Sigma, 106,000 U/mg), 5 μL of the TBABr-Nafion-N—CNT solution was pipetted onto the GC surface. For glucose determination using co-immobilized GOx (see FIG. 1), 10 mg GOx was added to 200 μL TBABr-Nafion-N—CNT solution and vortexed for 10 seconds before pipetting 5 μL of the mixture onto the GC surface. GC surfaces were covered to prevent contamination and allowed to dry for about 10 minutes. Upon drying, the N—CNT- and N—CNT/GOx-modified GC electrodes were immediately immersed into solution for use in electrochemical experiments. The N—CNTs appeared to be strongly adherent to the GC surface, as no N—CNTs dislodged upon immersion.

In addition to the GC working electrode, an Au wire counter electrode and Hg/Hg₂SO₄ (sat'd. K₂SO₄) reference electrode (CH Instruments, E°=+0.64 V vs. NHE) were used in all electrochemical measurements. All electrode potentials are reported vs. Hg/Hg₂SO₄. Electrochemical measurements were performed at room temperature (23±2° C.) using an AUTOLAB™ PGSTAT30 potentiostat interfaced with AUTOLAB™ GPES version 4.9 software. Electrodes were contained within a 125 mL volume, 5-neck glass cell containing 100 mL of 0.1 M Na₂HPO₄ at pH 6.00±0.03. Experiments were conducted under saturated O₂ conditions by flowing O₂ through the cell at all times. For rotating disk amperometry (RDE) experiments, a rotation rate (ω) of 1000 rpm was used.

Solutions of H₂O₂ (obtained commercially from Fisher Scientific, Waltham, Mass.) and D-glucose (obtained commercially from Sigma-Aldrich, Inc.) were injected into the cell in intervals of 30 seconds using an automated syringe pump (obtained commercially from New Era, Inc.). Solutions of both analytes and Na₂HPO₄ supporting electrolyte were prepared with ultrapure (>18.2 MΩ/cm) water.

In accordance with an embodiment, with reference to FIG. 1, the scheme as described above for sensing glucose is depicted schematically. As seen therein, a GC electrode (with a potential of −0.150 V with respect to an Hg/Hg₂SO₄ reference electrode) is provided which has been coated with N—CNTs using the process described above.

FIG. 1 provides a schematic depiction of the detection of H₂O₂ generated from GOx-glucose interaction at N—CNTs, resulting in glucose detection at N—CNTs. In the scheme of FIG. 1, GOx and N—CNTs are co-immobilized at a GC electrode, and glucose is introduced into the supporting electrolyte. As GOx oxidizes glucose to gluconolactone, H₂O₂ is produced stoichiometrically. The N—CNTs then catalytically decompose the generated H₂O₂, leading to a local increase in O₂, which is reduced at the N—CNTs to provide a measurable amperometric signal at −0.15 V.

Representative cyclic voltammograms (CVs) for oxygen reduction at both undoped CNT- and N—CNT-modified GC electrodes in 0.1 M Na₂HPO₄ are shown in FIG. 2. These CVs illustrate the catalytic nature of the N—CNTs, as oxygen is reduced at a much lower overpotential than that required for reduction at undoped CNTs, The catalytic activity of the N—CNTs toward oxygen reduction increases with increasing N content.

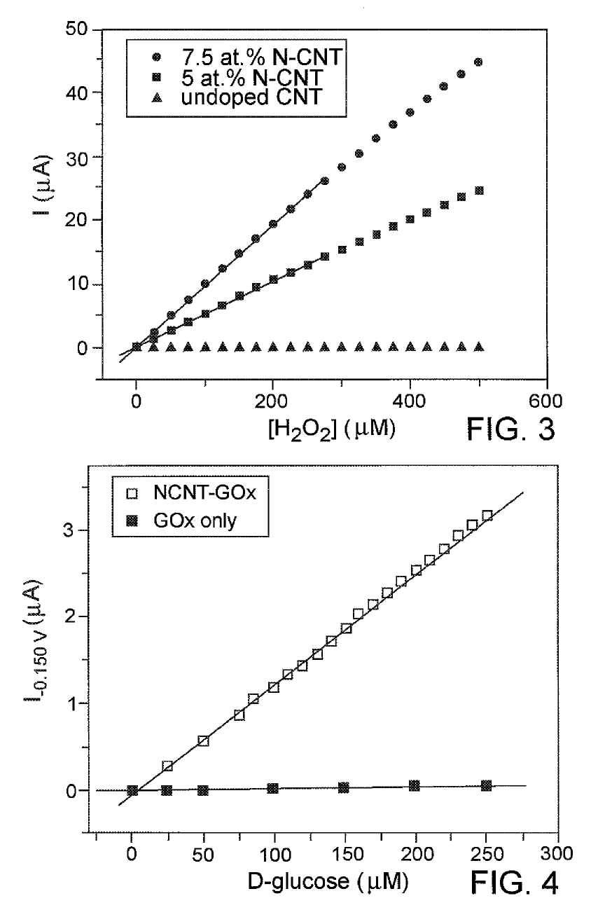

FIG. 3 depicts a response curve for an N—CNT modified GC electrode used to sense hydrogen peroxide added directly to the aqueous solution. FIG. 4 depicts a response curve for an N—CNT/GOx modified GC electrode used to sense D-glucose. The curve marked with triangles in FIG. 3 represents undoped CNTs used on the electrode, while the lower curve in FIG. 4 corresponds to the case where no N—CNTs are present on the electrode. The remaining curves in FIG. 3 represent examples using different amounts of N—CNT on the electrode, while the upper curve in FIG. 4 corresponds to the case where both GOx and N—CNTs are present on the electrode

As seen by the lower curve in FIG. 4, the electrode registers no response to the increasing concentration of D-glucose in the absence of N—CNTs, while the curve marked with triangles in FIG. 3 demonstrates that similar results are observed if the CNTs are undoped. On the other hand, in an embodiment, when N—CNTs are present, either alone or in combination with GOx, the response curves may be a linear function of D-glucose concentration.

In an embodiment, the fact that the signal scales in a linear fashion with concentration demonstrates the suitability of the electrode for D-glucose sensing, since this indicates that D-glucose concentration may be readily determined from the measured signal and the slope of the curve. It is also notable that the current is large at low potentials for the formation of hydrogen peroxide. The large slope of the curve indicates that the sensitivity of the system is very good (i.e., there is a large change in signal for a relatively small change in concentration of D-glucose). The electrode also affords very low (i.e., 100 nM) detection limits, making it ideal for physiological applications (such as, for example, the sensing of blood sugar levels).

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope of the present invention. Those with skill in the art will readily appreciate that embodiments in accordance with the present invention may be implemented in a very wide variety of ways. This application is intended to cover any adaptations or variations of the embodiments discussed herein. Therefore, it is manifestly intended that embodiments in accordance with the present invention be limited only by the claims and the equivalents thereof.

What is claimed is:

1. A method for detecting a composition, comprising:
providing a first composition that reacts with an oxidase to generate a second composition; and
detecting the first composition with nitrogen-doped nanocarbons.

2. The method of claim 1, wherein the first composition is detected with the nanocarbons by detecting the second composition.

3. The method of claim 1, wherein providing a first composition that reacts with an oxidase to generate a second composition comprises providing a first composition that reacts with an oxidase to generate hydrogen peroxide.

4. The method of claim 1, wherein the nanocarbons catalyze the decomposition of the second composition, and wherein the first composition is detected by monitoring the decomposition of the second composition.

5. The method of claim 1, wherein detecting the substrate includes determining the concentration of the substrate in solution.

6. The method of claim 1, wherein detecting the first composition with nitrogen-doped nanocarbons comprises detecting the first composition with nitrogen-doped carbon nanotubes (N—CNTs).

7. The method of claim 1, wherein providing a first composition comprises providing a first composition selected from the group consisting of pyruvate, lactate, glutamate, ascorbate and glucose.

8. The method of claim 1, wherein detecting the first composition with nitrogen-doped nanocarbons comprises detecting the first composition with nanocarbons disposed on a surface of a first electrode.

9. The method of claim 8, wherein detecting the first composition further comprises detecting the first composition with the first electrode, a counter electrode, and a reference electrode.

10. The method of claim 8, wherein detecting the first composition further comprises detecting the first composition with a glassy carbon electrode, an Au wire electrode, and a saturated Hg/Hg₂SO₄ electrode.

11. A device for sensing a substrate, comprising:
a first electrode for sensing a first composition that reacts with an oxidase to generate a second composition; and
a plurality of nitrogen-doped nanocarbons disposed on a surface of said first electrode.

12. The device of claim 11, wherein the nanocarbons catalyze the decomposition of the second composition, and wherein the first electrode senses the first composition by monitoring the decomposition of the second composition.

13. The device of claim 11, wherein the nanocarbons are nitrogen-doped carbon nanotubes (N—CNTs).

14. The device of claim 11, wherein the first electrode is a carbon electrode.

15. The device of claim 14, wherein the first electrode is a glassy carbon electrode.

16. The device of claim 11, wherein the nanocarbons are disposed in a matrix comprising an ion exchange polymer.

17. The device of claim 16, wherein the ion exchange polymer is a persulfonated ion exchange polymer.

18. The device of claim 17, wherein the ion exchange polymer is modified with tetrabutylammonium bromide.

19. The device of claim 16, wherein the matrix further comprises a portion of the oxidase.

20. The device of claim 16, wherein the matrix is a film in which the oxidase is co-immobilized with the nanocarbons.

21. The device of claim 11, wherein the nanocarbons are N—CNTs having average nominal lengths within the range of about 5 μm to about 15 μm.

22. The device of claim 11, wherein the nanocarbons are N—CNTs having average nominal diameters within the range of about 10 μm to about 50 μm.

23. The device of claim 11, wherein the device further comprises a counter electrode and a reference electrode.

24. The device of claim 23, wherein the counter electrode is an Au wire electrode, and wherein the reference electrode is a saturated Hg/HgSO₄ electrode.

25. A substrate sensing system, comprising:
a first electrode for sensing a first composition that reacts with an oxidase to generate a second composition;
a plurality of nitrogen-doped nanocarbons disposed on a surface of said first electrode; and
at least one electrical component for processing a signal or current generated by the second composition and detected by the first electrode, wherein the signal or current detected is indicative of a concentration of the first composition.
